(19) United States
(12) Patent Application Publication
(10) Pub. No.: US 2014/0303475 A1
(43) Pub. Date: Oct. 9, 2014

(54) Device to Aid in Diagnosing Infiltration or Extravasation in Animalia Tissue

(71) Applicants: Garret T. Bonnema, Williamsburg, VA (US); Javier A. Garriz, Williamsburg, VA (US); Matthew S. Alley, Williamsburg, VA (US); Gary P. Warren, Williamsburg, VA (US); William J. Naramore, Williamsburg, VA (US); ivWatch, LLC, Williamsburg, VA (US)

(72) Inventors: Gary P. Warren, Williamsburg, VA (US); Matthew S. Alley, Sandston, VA (US); Javier A. Garriz, Hampton, VA (US); William J. Naramore, Yorktown, VA (US); Garret T. Bonnema, Williamsburg, VA (US)

(73) Assignee: ivWatch, LLC, Williamsburg, VA (US)

(21) Appl. No.: 14/245,569

(22) Filed: Apr. 4, 2014

Related U.S. Application Data
(60) Provisional application No. 61/809,651, filed on Apr. 8, 2013.

(51) Int. Cl. A61M 5/168; A61B 5/00; A61B 5/02

(52) U.S. Cl. CPC A61M 5/16836; A61B 5/02042; A61B 5/441
USPC 600/407

(57) ABSTRACT

An aid to diagnosing at least one of infiltration and extravasation in Animalia tissue includes an optics bench and a controller. The optics bench includes a light emitting diode and a photodiode. The light emitting diode is configured to emit a first light signal, and the photodiode is configured to detect a second light signal. The second light signal includes a portion of the first light signal that is at least one of reflected, scattered and redirected from the Animalia tissue. The controller includes a processor, volatile memory and non-volatile memory. The non-volatile memory stores a sequence of values that correspond to the second light signal detected by the photodiode. The processor and volatile memory analyze the sequence of values according to an algorithm.

### CROSS-REFERENCES TO RELATED APPLICATIONS

This application claims the priority of U.S. Provisional Application No. 61/809,651, filed 8 Apr. 2013, which is hereby incorporated by reference in its entirety.

### STATEMENT REGARDING FEDERALLY SPONSORED RESEARCH OR DEVELOPMENT

Not Applicable

### BACKGROUND OF THE INVENTION

FIGS. 8A and 8B show a typical arrangement for intravascular infusion. As the terminology is used herein, “intravascular” preferably refers to being situated in, occurring in, or being administered by entry into a blood vessel, thus “intravascular infusion” preferably refers to introducing a fluid or infusate into a blood vessel. Intravascular infusion accordingly encompasses both intravenous infusion (administering a fluid into a vein) and intra-arterial infusion (administering a fluid into an artery).

A cannula 20 is typically used for administering fluid via a subcutaneous blood vessel V. Typically, cannula 20 is inserted through skin S at a cannulation or cannula insertion site N and punctures the blood vessel V, for example, the cephalic vein, basilica vein, median cubital vein, or any suitable vein for an intravenous infusion. Similarly, any suitable artery may be used for an intra-arterial infusion.

Cannula 20 typically is in fluid communication with a fluid source 22. Typically, cannula 20 includes an extracorporeal connector, e.g., a hub 20a, and a transcutaneous sleeve 20b. Fluid source 22 typically includes one or more sterile containers that hold the fluid(s) to be administered. Examples of typical sterile containers include plastic bags, glass bottles or plastic bottles.

An administration set 30 typically provides a sterile conduit for fluid to flow from fluid source 22 to cannula 20. Typically, administration set 30 includes tubing 32, a drip chamber 34, a flow control device 36, and a cannula connector 38. Tubing 32 is typically made of polypropylene, nylon, or another flexible, strong and inert material. Drip chamber 34 typically permits the fluid to flow one drop at a time for reducing air bubbles in the flow. Tubing 32 and drip chamber 34 are typically transparent or translucent to provide a visual indication of the flow. Typically, flow control device 36 is positioned upstream from drip chamber 34 for controlling fluid flow in tubing 32. Roller clamps and Dial-A-Flo®, manufactured by Hospira, Inc. (Lake Forest, Ill., US), are examples of typical flow control devices. Typically, cannula connector 38 and hub 20a provide a leak-proof coupling through which the fluid may flow. Luer-Lok™, manufactured by Becton, Dickinson and Company (Franklin Lakes, N.J., US), is an example of a typical leak-proof coupling.

Administration set 30 may also include at least one of a clamp 40, an injection port 42, a filter 44, or other devices. Typically, clamp 40 pinches tubing 32 to cut-off fluid flow. Injection port 42 typically provides an access port for administering medicine or another fluid via cannula 20. Filter 44 typically purifies and/or treats the fluid flowing through administration set 30. For example, filter 44 may strain contaminants from the fluid.

An infusion pump 50 may be coupled with administration set 30 for controlling the quantity or the rate of fluid flow to cannula 20. The Alaris® System manufactured by CareFusion Corporation (San Diego, Calif., US), BodyGuard® Infusion Pumps manufactured by CMA America, L.L.C. (Golden, Colo., US), and Flo-Gard® Volumetric Infusion Pumps manufactured by Baxter International Inc. (Deerfield, Ill., US) are examples of typical infusion pumps.

Intravenous infusion or therapy typically uses a fluid (e.g., infusate, whole blood, or blood product) to correct an electrolyte imbalance, to deliver a medication, or to elevate a fluid level. Typical infusates predominately consist of sterile water with electrolytes (e.g., sodium, potassium, or chloride), calories (e.g., dextrose or total parenteral nutrition), or medications (e.g., anti-infectives, anticonvulsants, antihyperuricemic agents, cardiovascular agents, central nervous system agents, chemotherapy drugs, coagulation modifiers, gastrointestinal agents, or respiratory agents). Examples of medications that are typically administered during intravenous therapy include acyclovir, allopurinol, amikacin, aminophylline, amiodarone, amphotericin B, ampicillin, carboplatin, cefazolin, cefotaxime, cefuroxime, ciprofloxacin, cisplatin, clindamycin, cyclophosphamide, diazepam, docetaxel, dopamine, doxorubicin, doxycycline, erythromycin, etoposide, fentanyl, fluorouracil, furosemide, ganciclovir, gemcitabine, gentamicin, heparin, imipenem, irinotecan, lorazepam, magnesium sulfate, meropenem, methotrexate, methylprednisolone, midazolam, morphine, nafcillin, ondansetron, paclitaxel, pentamidine, phenobarbital, phenytoin, piperacillin, promethazine, sodium bicarbonate, ticarcillin, tobramycin, topotecan, vancomycin, vinblastine and vincristine. Transfusions and other processes for donating and receiving whole blood or blood products (e.g., albumin and immunoglobulin) also typically use intravenous infusion.

Unintended infusing typically occurs when fluid from cannula 20 escapes from its intended vein/artery. Typically, unintended infusing causes an abnormal amount of the fluid to diffuse or accumulate in perivascular tissue P and may occur, for example, when (i) cannula 20 causes a vein/artery to rupture; (ii) cannula 20 improperly punctures the vein/artery; (iii) cannula 20 backs out of the vein/artery; (iv) cannula 20 is improperly sized; (v) infusion pump 50 administers fluid at an excessive flow rate; or (vi) the infusate increases permeability of the vein/artery. As the terminology is used herein, “tissue” preferably refers to an association of cells, intercellular material and/or interstitial compartments, and “perivascular tissue” preferably refers to cells, intercellular material and/or interstitial compartments that are in the general vicinity of a blood vessel and may become unintentionally infused with fluid from cannula 20. Unintended infusing of a non-vesicant fluid is typically referred to as “infiltration,” whereas unintended infusing of a vesicant fluid is typically referred to as “extravasation.”

The symptoms of infiltration or extravasation typically include blanching or discoloration of the skin S, edema, pain, or numbness. The consequences of infiltration or extravasation typically include skin reactions (e.g., blisters), nerve compression, compartment syndrome, or necrosis. Typical treatment for infiltration or extravasation includes applying warm or cold compresses, elevating an affected limb, administering hyaluronidase, phentolamine, sodium thiosulfate or dexrazoxane, fasciotomy, or amputation.

### BRIEF SUMMARY OF THE INVENTION

Embodiments according to the present invention include a device for aiding in diagnosing at least one of infiltration and extravasation in Animalia tissue. A cable couples the device with a sensor disposed on an epidermis of the Animalia tissue. The device includes a housing that has an exterior surface and defines an interior space. The device also includes a keypad disposed on the exterior surface, an optics bench disposed in the interior space, a notification section disposed on the exterior surface, a processor disposed in the interior space, non-volatile memory disposed in the interior space, and an input/output section communicating with the processor. The processor communicates with the keypad, the optics bench and the notification section. The non-volatile memory communicates with the processor. The optics bench includes a light emitting diode configured to emit a first near-infrared signal and a photodiode configured to detect a second near-infrared signal. The second near-infrared signal includes a portion of the first near-infrared signal that is at least one of reflected, scattered and redirected from the Animalia tissue.

Other embodiments according to the present invention include a device to aid in diagnosing at least one of infiltration and extravasation in Animalia tissue. The device includes an optics bench, a controller and an indicator coupled to the processor. The optics bench includes a light emitting diode and a photodiode. The light emitting diode is configured to emit a first light signal, and the photodiode is configured to detect a second light signal. The second light signal includes a portion of the first light signal that is at least one of reflected, scattered and redirected from the Animalia tissue. The controller includes volatile memory and a processor. The volatile memory is configured to store a sequence of values corresponding to the second light signal detected by the photodiode. The processor is configured to analyze the sequence of values stored in the volatile memory. The indicator is configured to output a notice regarding at least one of infiltration and extravasation in the Animalia tissue.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features, principles, and methods of the invention.

FIG. 1 illustrates a system according to the present disclosure for aiding in diagnosing at least one of infiltration and extravasation in Animalia tissue.

FIG. 2 is a schematic view illustrating an electromagnetic radiation sensor of the system shown in FIG. 1. The electromagnetic radiation sensor is shown contiguously engaging Animalia skin, e.g., human skin.

FIGS. 3A-3C are schematic cross-section views demonstrating how an anatomical change over time in perivascular tissue impacts the system shown in FIG. 1.

FIGS. 4A-4C are perspective views illustrating a patient monitoring device of the system shown in FIG. 1.

FIGS. 5A and 5B are perspective views illustrating a transceiver according to one embodiment of the patient monitoring device shown in FIGS. 4A-4C.

FIG. 6 is a schematic diagram illustrating components according to one embodiment of the patient monitoring device shown in FIGS. 4A-4C.

FIGS. 7A-7G schematically illustrate a circuit diagram of a controller according to one embodiment of the patient monitoring device shown in FIGS. 4A-6. The circuit diagram has been broken into parts and shown on multiple sheets to facilitate understanding; matching letters indicate the connections for the breaks across the sheets.

FIGS. 8A-8C schematically illustrate a circuit diagram of an optics bench according to one embodiment of the patient monitoring device shown in FIGS. 4A-6. The circuit diagram has been broken into parts and shown on multiple sheets to facilitate understanding; matching letters indicate the connections for the breaks across the sheets.

FIG. 9A is a schematic view illustrating a typical set-up for infusion administration.

FIG. 9B is a schematic view illustrating a subcutaneous detail of the set-up shown in FIG. 9A.

In the figures, the thickness and configuration of components may be exaggerated for clarity. The same reference numerals in different figures represent the same component.

### DETAILED DESCRIPTION OF THE INVENTION

The following description and drawings are illustrative and are not to be construed as limiting. Numerous specific details are described to provide a thorough understanding of the disclosure. However, in certain instances, well-known or conventional details are not described in order to avoid obscuring the description.

Reference in this specification to “one embodiment” or “an embodiment” means that a particular feature, structure, or characteristic described in connection with the embodiment is included in at least one embodiment according to the disclosure. The appearances of the phrases “one embodiment” or “other embodiments” in various places in the specification are not necessarily all referring to the same embodiment, nor are separate or alternative embodiments mutually exclusive of other embodiments. Moreover, various features are described that may be exhibited by some embodiments and not by others. Similarly, various features are described that may be included in some embodiments but not other embodiments.

The terms used in this specification generally have their ordinary meanings in the art, within the context of the disclosure, and in the specific context where each term is used. Certain terms in this specification may be used to provide additional guidance regarding the description of the disclosure. It will be appreciated that a feature may be described more than one-way.

Alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term.

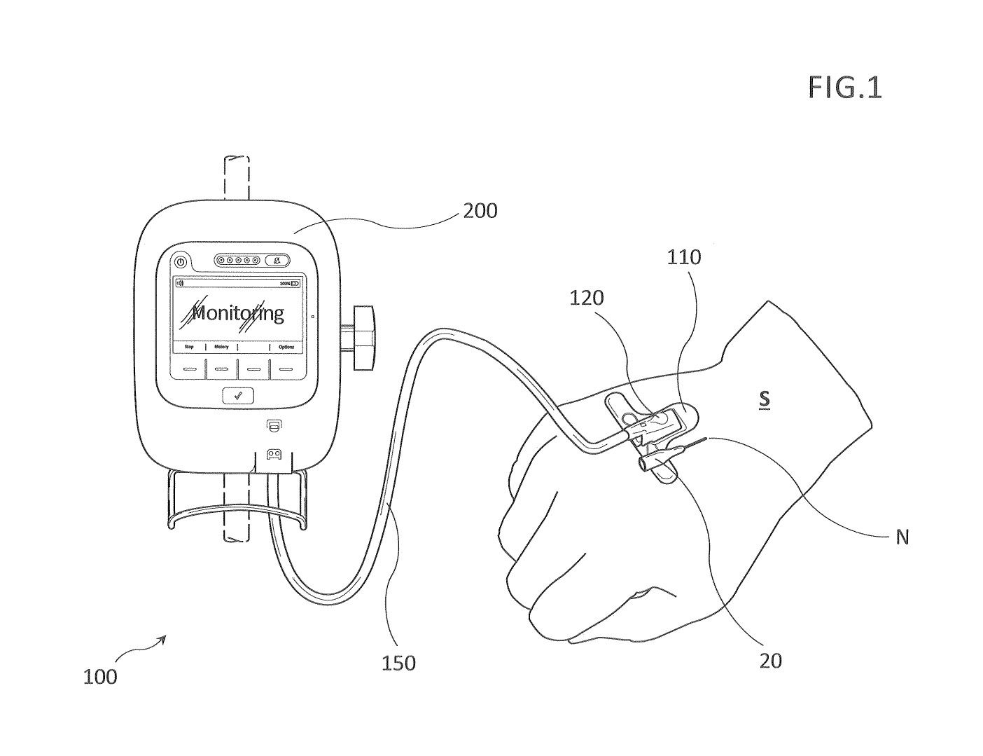

FIG. 1 shows a system 100 to preferably aid in diagnosing at least one of infiltration and extravasation in Animalia tissue. Preferably, system 100 includes a dressing 110, an electromagnetic radiation sensor 120, a sensor cable 150, and a patient monitoring device 200. Dressing 110 preferably couples electromagnetic radiation sensor 120 with the skin S. Preferably, electromagnetic radiation sensor 120 is arranged to overlie a target area of the skin S. As the terminology is used herein, “target area” preferably refers to a portion of a patient's skin that is generally proximal to where an infusate is being administered and frequently proximal to the cannulation site N. Preferably, the target area overlies the perivascular tissue P. According to one embodiment, adhesion preferably is used to couple electromagnetic radiation sensor 120 to the skin S. Preferably, the skin S and dressing 110 have generally similar viscoelastic characteristics such that both respond in a generally similar manner to stress and strain. According to other embodiments, any suitable coupling may be used that preferably minimizes relative movement between electromagnetic radiation sensor 120 and the skin S.

Dressing 110 preferably includes different arrangements that permit electromagnetic radiation sensor 120 to be coupled, decoupled and recoupled, e.g., facilitating multiple independent uses with one or a plurality of dressings 110. As the terminology is used herein, “arrangement” preferably refers to a relative configuration, formation, layout or disposition of dressing 110 and electromagnetic radiation sensor 120. Preferably, dressing 110 includes a first arrangement that retains electromagnetic radiation sensor 120 relative to the skin S for monitoring infiltration or extravasation during an infusion with cannula 20. A second arrangement of dressing 110 preferably releases electromagnetic radiation sensor 120 from the first arrangement. Accordingly, electromagnetic radiation sensor 120 may be decoupled from a singular dressing 110 in the second arrangement, e.g., during patient testing or relocation, and subsequently recoupled in the first arrangement of the singular dressing 110 such that a relationship between electromagnetic radiation sensor 120 and the skin S is generally repeatable. Electromagnetic radiation sensor 120 may also be coupled to a first dressing 110 in the first arrangement, decoupled from the first dressing 110 in the second arrangement, and subsequently coupled to a second dressing 110 in the first arrangement.

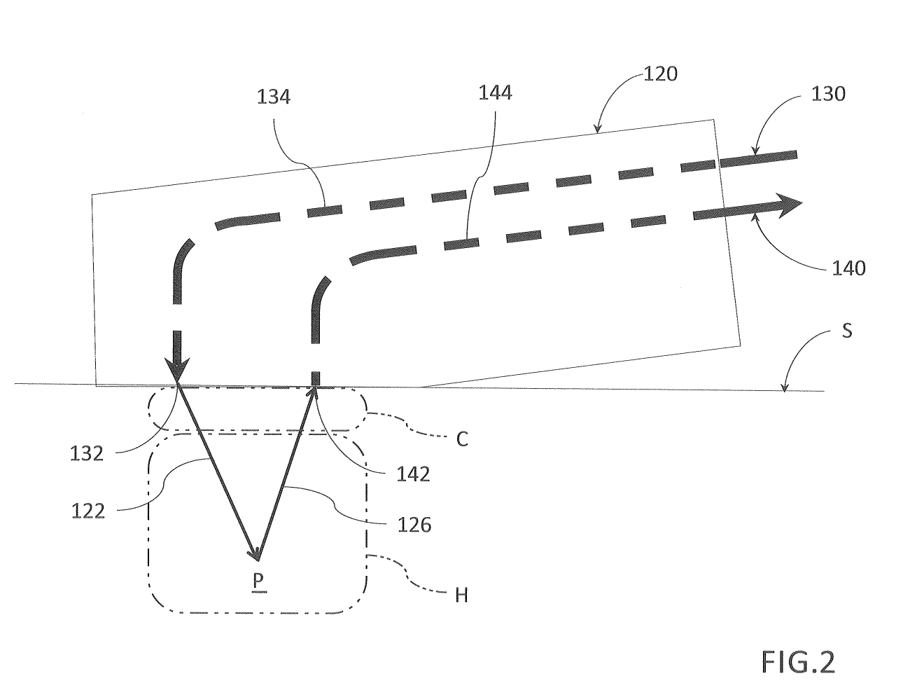

FIG. 2 shows an electromagnetic radiation sensor 120 that preferably includes an anatomic sensor. As the terminology is used herein, “anatomic” preferably refers to the structure of an Animalia body and an “anatomic sensor” preferably is concerned with sensing a change over time of the structure of the Animalia body. By comparison, a physiological sensor is concerned with sensing the functions or activities of an Animalia body, e.g., pulse or blood chemistry, at a point in time.

Electromagnetic radiation sensor 120 preferably emits and collects transcutaneous electromagnetic radiation. Preferably, electromagnetic radiation sensor 120 emits electromagnetic radiation 122 and collects electromagnetic radiation 126. Emitted electromagnetic radiation 122 preferably passes through the target area of the skin S into relatively shallow tissue, e.g., cutaneous tissue C. Preferably, emitted electromagnetic radiation 122 propagates toward the perivascular tissue P in relatively deep tissue, e.g., hypodermis H. Collected electromagnetic radiation 126 preferably includes a portion of emitted electromagnetic radiation 122 that is at least one of specularly reflected, diffusely reflected (e.g., due to elastic or inelastic scattering), fluoresced (e.g., due to endogenous or exogenous factors), or otherwise redirected from the perivascular tissue P before passing through the target area of the skin S.

Electromagnetic radiation sensor 120 preferably includes waveguides to transmit emitted and collected electromagnetic radiation 122 and 126. As the terminology is used herein, “waveguide” preferably refers to a duct, pipe, fiber, or other device that generally confines and directs the propagation of electromagnetic radiation along a path. Preferably, an emission waveguide 130 includes an emitter face 132 for emitting electromagnetic radiation 122 and a detection waveguide 140 includes a detector face 142 for collecting electromagnetic radiation 126. According to one embodiment, emission waveguide 130 preferably includes a set of emission optical fibers 134 and detection waveguide 140 preferably includes a set of detection optical fibers 144. Individual emission and detection optical fibers 134 and 144 preferably each have an end face. Preferably, an aggregation of end faces of emission optical fibers 134 forms emitter face 132 and an aggregation of end faces of detection optical fibers 144 forms detector face 142.

The transcutaneous electromagnetic radiation signals emitted by electromagnetic radiation sensor 120 preferably are not harmful to an Animalia body. Preferably, the wavelength of emitted electromagnetic radiation 122 is longer than at least approximately 400 nanometers. The frequency of emitted electromagnetic radiation 102 therefore is no more than approximately 750 terahertz. According to one embodiment, emitted electromagnetic radiation 122 is in the visible radiation (light) or infrared radiation portions of the electromagnetic spectrum. Preferably, emitted electromagnetic radiation 122 is in the near infrared portion of the electromagnetic spectrum. As the terminology is used herein, “near infrared” preferably refers to electromagnetic radiation having wavelengths between approximately 600 nanometers and approximately 2,100 nanometers. These wavelengths correspond to a frequency range of approximately 500 terahertz to approximately 145 terahertz. A desirable range in the near infrared portion of the electromagnetic spectrum preferably includes wavelengths between approximately 800 nanometers and approximately 1,050 nanometers. These wavelengths correspond to a frequency range of approximately 375 terahertz to approximately 285 terahertz. According to other embodiments, electromagnetic radiation sensor 120 may emit electromagnetic radiation signals in shorter wavelength portions of the electromagnetic spectrum, e.g., ultraviolet light, X-rays or gamma rays, preferably when radiation intensity and/or signal duration are such that tissue harm is minimized.

Emitted and collected electromagnetic radiation 122 and 126 preferably share one or more wavelengths. According to one embodiment, emitted and collected electromagnetic radiation 122 and 126 preferably share a single peak wavelength, e.g., approximately 940 nanometers (approximately 320 terahertz). As the terminology is used herein, “peak wavelength” preferably refers to an interval of wavelengths including a spectral line of peak power. The interval preferably includes wavelengths having at least half of the peak power. Preferably, the wavelength interval is +/−approximately 20 nanometers with respect to the spectral line. According to other embodiments, emitted and collected electromagnetic radiation 122 and 126 preferably share a plurality of peak wavelengths, e.g., approximately 940 nanometers and approximately 650 nanometers (approximately 460 terahertz). According to other embodiments, a first one of emitted and collected electromagnetic radiation 122 and 126 preferably spans a first range of wavelengths, e.g., from approximately 600 nanometers to approximately 1000 nanometers. This wavelength range corresponds to a frequency range from approximately 500 terahertz to approximately 300 terahertz. A second one of emitted and collected electromagnetic radiation 122 and 126 preferably shares with the first range a single peak wavelength, a plurality of peak wavelengths, or a second range of wavelengths. Preferably, an optical power analysis at the wavelength(s) shared by emitted and collected electromagnetic radiation 122 and 126 provides an indication of anatomical change over time in the perivascular tissue P.

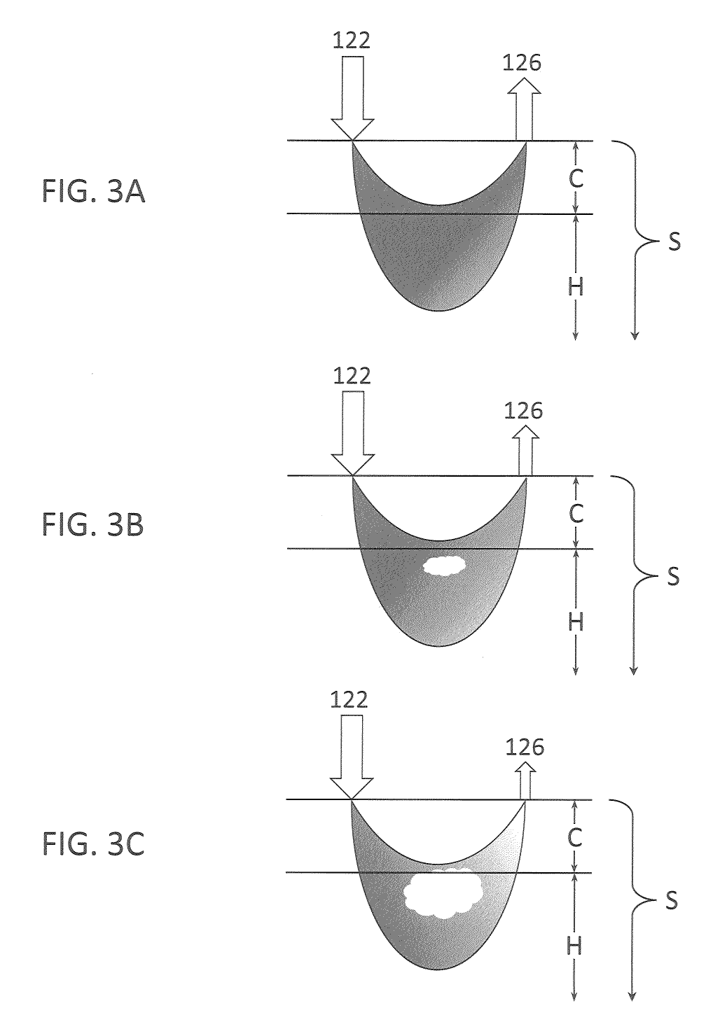

FIGS. 3A-3C schematically illustrate how an infiltration/extravasation event preferably evolves. FIG. 3A shows the skin S prior to an infiltration/extravasation event. Preferably, the skin S includes the cutaneous tissue C, e.g., stratum corneum, epidermis and/or dermis, overlying subcutaneous tissue, e.g., the hypodermis H. Blood vessels V suitable for intravenous therapy typically are disposed in the hypodermis H. FIG. 3B shows an infusate F beginning to accumulate in the perivascular tissue P. Accumulation of the infusate F typically begins in the hypodermis H, but may also begin in the cutaneous tissue C or at an interface of the hypodermis H with the cutaneous tissue C. FIG. 3C shows additional accumulation of the infusate F in the perivascular tissue P. Typically, the additional accumulation extends further in the hypodermis H but may also extend into the cutaneous tissue C. According to one embodiment, an infiltration/extravasation event generally originates and/or occurs in proximity to the blood vessel V, e.g., as illustrated in FIGS. 3A-3C. According to other embodiments, an infiltration/extravasation event may originate and/or occur some distance from the blood vessel V, e.g., if pulling on the cannula 20 or administration set 30 causes the cannula outlet to become displaced from the blood vessel V.

FIGS. 3A-3C also schematically illustrate the relative power of emitted and collected electromagnetic radiation 122 and 126. Preferably, emitted electromagnetic radiation 122 enters the skin S, electromagnetic radiation propagates through the skin S, and collected electromagnetic radiation 126 exits the skin S. Emitted electromagnetic radiation 122 is schematically illustrated with an arrow directed toward the skin S and collected electromagnetic radiation 126 is schematically illustrated with an arrow directed away from the skin S. Preferably, the relative sizes of the arrows correspond to the relative powers of emitted and collected electromagnetic radiation 122 and 126. The propagation is schematically illustrated with crescent shapes that preferably include the predominant electromagnetic radiation paths through the skin S from emitted electromagnetic radiation 122 to collected electromagnetic radiation 126. Stippling in the crescent shapes schematically illustrates a distribution of electromagnetic radiation power in the skin S with relatively lower power generally indicated with less dense stippling and relatively higher power generally indicated with denser stippling.

The power of collected electromagnetic radiation 126 preferably is impacted by the infusate F accumulating in the perivascular tissue P. Prior to the infiltration/extravasation event (FIG. 3A), the power of collected electromagnetic radiation 126 preferably is a fraction of the power of emitted electromagnetic radiation 122 due to electromagnetic radiation scattering and absorption by the skin S. Preferably, the power of collected electromagnetic radiation 126 changes with respect to emitted electromagnetic radiation 122 in response to the infusate F accumulating in the perivascular tissue P (FIGS. 3B and 3C). According to one embodiment, emitted and collected electromagnetic radiation 122 and 126 include near infrared electromagnetic radiation. The power of collected electromagnetic radiation 126 preferably decreases due to scattering and/or absorption of near infrared electromagnetic radiation by the infusate F. The compositions of most infusates typically are dominated by water. Typically, water has different absorption and scattering coefficients as compared to the perivascular tissue P, which contains relatively strong near infrared energy absorbers, e.g., blood. At wavelengths shorter than approximately 700 nanometers (approximately 430 terahertz), absorption coefficient changes preferably dominate due to absorption peaks of blood. Preferably, scattering coefficient changes have a stronger influence than absorption coefficient changes for wavelengths between approximately 800 nanometers (approximately 375 terahertz) and approximately 1,300 nanometers (approximately 230 terahertz). In particular, propagation of near infrared electromagnetic radiation in this range preferably is dominated by scattering rather than absorption because scattering coefficients have a larger magnitude than absorption coefficients. Absorption coefficient changes preferably dominate between approximately 1,300 nanometers and approximately 1,500 nanometers (approximately 200 terahertz) due to absorption peaks of water. Therefore, the scattering and/or absorption impact of the infusate F accumulating in the perivascular tissue P preferably is a drop in the power signal of collected electromagnetic radiation 126 relative to emitted electromagnetic radiation 122. According to other embodiments, a rise in the power signal of collected electromagnetic radiation 126 relative to emitted electromagnetic radiation 122 preferably is related to infusates with different scattering and absorption coefficients accumulating in the perivascular tissue P. Thus, the inventors discovered, inter alio, that fluid changes in perivascular tissue P over time, e.g., due to an infiltration/extravasation event, preferably are indicated by a change in the power signal of collected electromagnetic radiation 126 with respect to emitted electromagnetic radiation 122.

Electromagnetic radiation sensor 120 preferably aids healthcare givers in identifying infiltration/extravasation events. Preferably, changes in the power signal of collected electromagnetic radiation 126 with respect to emitted electromagnetic radiation 122 alert a healthcare giver to perform an infiltration/extravasation evaluation. The evaluation that healthcare givers perform to identify infiltration/extravasation events typically includes palpitating the skin S in the vicinity of the target area, observing the skin S in the vicinity of the target area, and/or comparing limbs that include and do not include the target area of the skin S.

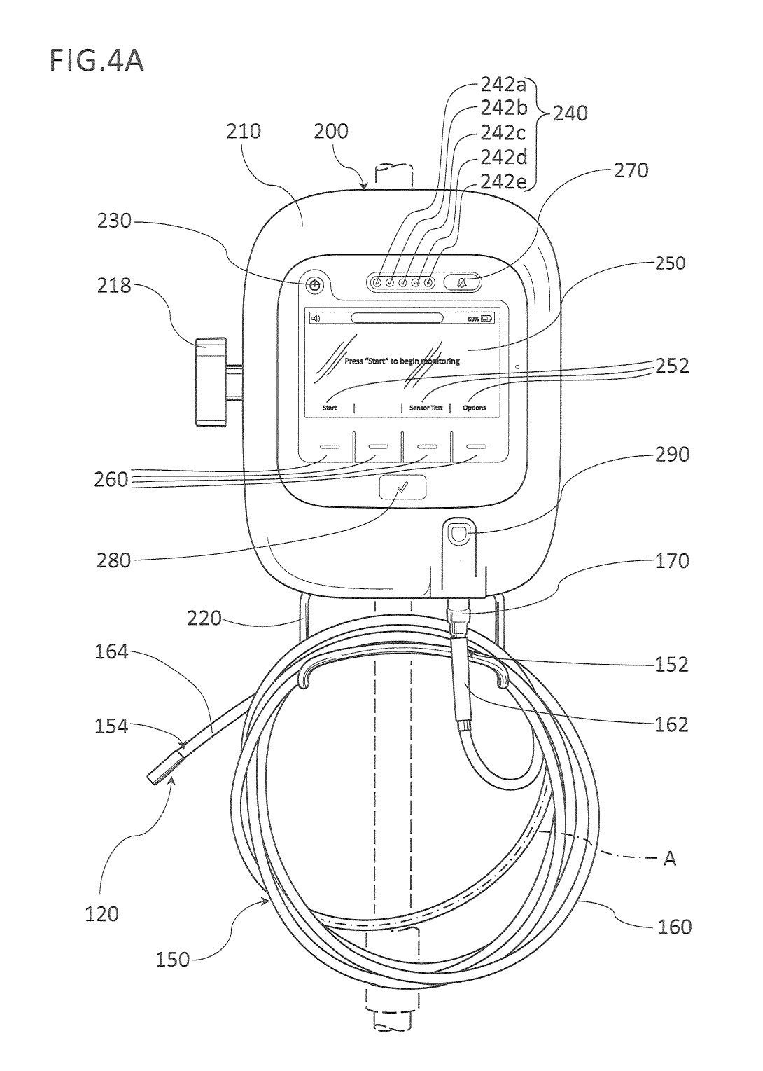

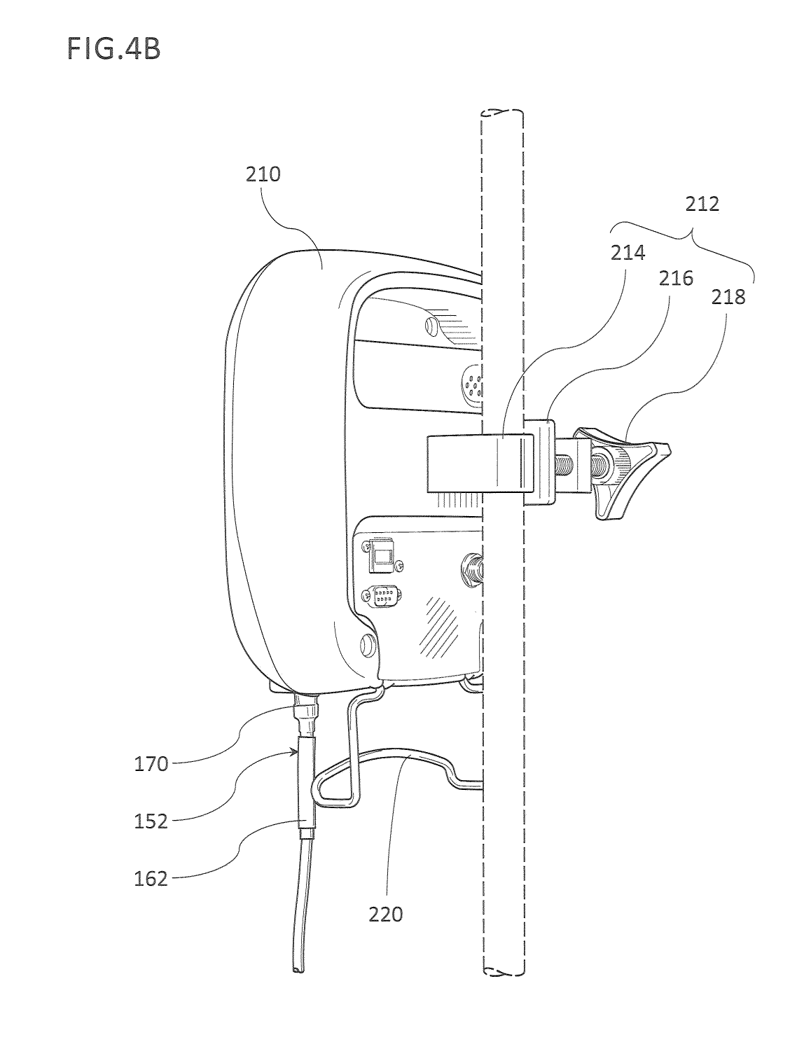

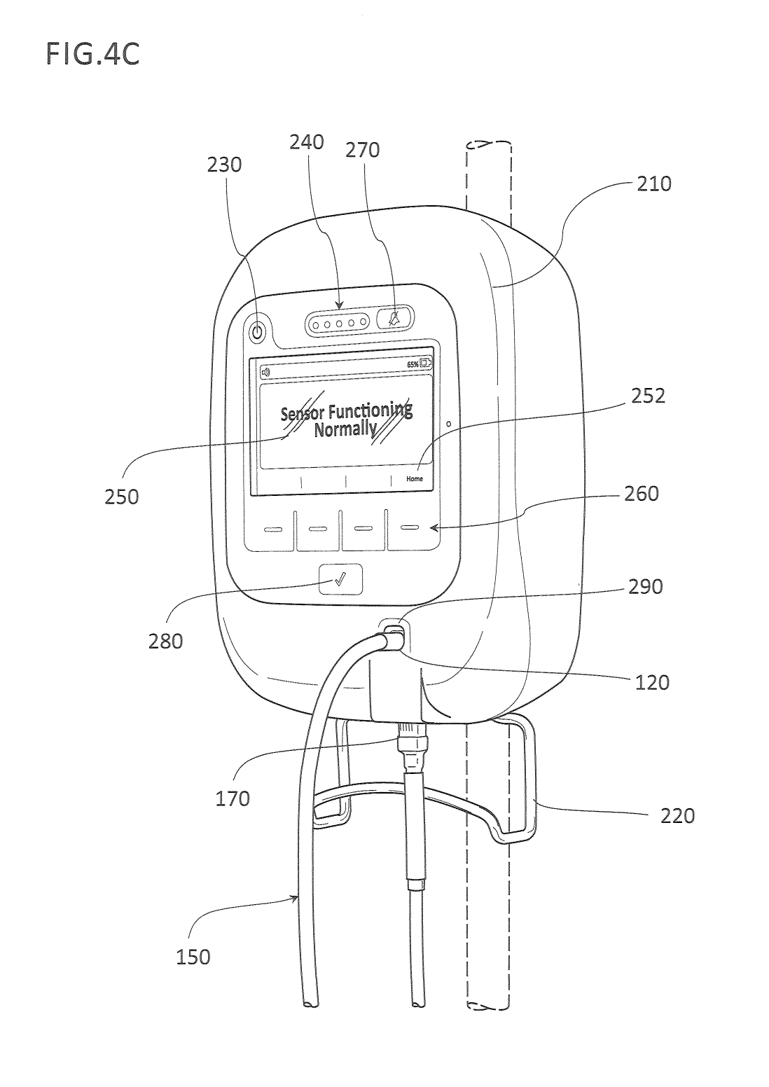

Sensor cable 150 preferably provides transmission paths for first and second light signals between patient monitoring device 200 and electromagnetic radiation sensor 120. According to one embodiment, emission optical fiber and detection optical fiber sets 134 and 144 preferably extend in sensor cable 150 along an axis A between first and second ends 152 and 154. Preferably, first end 152 is proximate to patient monitoring device 200 and second end 154 is proximate to electromagnetic radiation sensor 120. A sheath 160 preferably cinctures emission and detection optical fiber sets 134 and 144 along the axis A between first and second ends 152 and 154. Preferably, sheath 160 includes a first end 162 coupled to a plug 170 (FIGS. 4A-4C) and includes a second end 164 coupled to electromagnetic radiation sensor 120.

FIGS. 4A-4C illustrate the exterior of patient monitoring device 200 according to one embodiment of the present disclosure. Patient monitoring device 200 preferably includes a housing 210 supported on a pole by a clamp 212. Preferably, housing 210 includes an exterior surface and defines an interior space. According to one embodiment, clamp 212 preferably is disposed on the exterior of housing 210 and includes a fixed jaw 214 and a moving jaw 216. An actuator 218, e.g., a knob and threaded rod, preferably displaces moving jaw 216 relative to fixed jaw 214 for gripping and releasing clamp 212 with respect to the pole. Preferably, a bail 220 is coupled to housing 210 for capturing sensor cable 150, e.g., when dressing 110 is in the second arrangement. According to the embodiment shown in FIGS. 4A-4C, bail 220 includes a hook coupled to housing 210 at a plurality of junctures. According to other embodiments, bail 220 may be coupled to housing 210 at a single juncture or a basket or net slung from housing 210 may be used to capture at least a portion of sensor cable 150.

Patient monitoring device 200 preferably includes a number of features disposed on the exterior of housing 210. Preferably, patient monitoring device 200 includes a power button 230, an indicator set 240, a display 250, a set of soft keys 260, a mute button 270, a check button 280 and a test port 290. According to the embodiment shown in FIG. 4A, these features preferably are disposed on the front of housing 210. Pressing power button 230 preferably turns ON and OFF patient monitoring device 200.

Patient monitoring device 200 preferably provides status reports of varying detail. Preferably, indicator set 240 provides a basic status report and display 250 provides a more detailed status report. According to one embodiment, indicator set 240 includes a set of multi-color light emitting diodes 242a-242e providing a visible indication of one of three states of patient monitoring device 200. A first state of patient monitoring device 200 preferably includes all of multi-color light emitting diodes 242a-242e illuminating a first color, e.g., green. Preferably, the first state is characterized by actively monitoring for indications of infiltration or extravasation without identifying a cause for alerting a healthcare giver to evaluate the patient. A second state of patient monitoring device 200 preferably includes all of multi-color light emitting diodes 242a-242e illuminating a second color, e.g., yellow. Preferably, the second state is characterized by identifying a cause for alerting the healthcare giver to evaluate the operation of system 100 with respect to the patient. For example, the second state may be indicated if operation of system 100 is being disrupted because the patient is pulling on sensor cable 150. A third state of patient monitoring device 200 preferably includes all of multi-color light emitting diodes 242a-242e illuminating a third color, e.g., red. Preferably, the third state is characterized by patient monitoring device 200 alerting the healthcare giver to perform an infiltration or extravasation evaluation. According to other embodiments, the number as well as color(s) of multi-color light emitting diodes 242a-242e that are illuminated may provide information regarding, for example, duration or intensity of an event that is cause for alerting a healthcare giver.

Display 250 preferably provides detailed information regarding the use, status, and alarms of patient monitoring device 200. Preferably, display 250 includes color, alphanumeric characters, graphs, icons and images to convey set-up and operating instructions, system maintenance and malfunction notices, system configuration statements, healthcare giver alerts, historical records, etc. According to one embodiment, display 250 preferably displays individual labels 252 describing a function assigned to a corresponding soft key 260. According to other embodiments, display 250 preferably facilitates quantifying with precision when an identifiable event occurred, its duration, its magnitude, whether an alert was issued, and the corresponding type of alert.

Mute button 270 and check button 280 preferably are hard keys having regularly assigned functions. Preferably, mute button 270 temporarily silences an audible alarm. According to one embodiment, a healthcare giver preferably silences the audible alarm while performing an infiltration/extravasation evaluation. Preferably, the function of mute button 270 is temporary because disabling rather than silencing the audible alarm may be detrimental to the future effectiveness of patient monitoring device 200. Check button 280 preferably includes one or more regularly assigned functions, e.g., registering periodic evaluations of the insertion site N. According to one embodiment, check button 280 is preferably pressed each time a healthcare giver performs an evaluation of the insertion site N. Preferably, the evaluation is registered in a historical record maintained by patient monitoring device 200. According to other embodiments, the historical record may be reviewed on display 250 and/or the historical record may be transferred off patient monitoring device 200 to a recordkeeping system that maintains a generally comprehensive chronicle of the patient's treatment(s).

Patient monitoring device 200 preferably includes a test arrangement for verifying the operation and calibration of system 100. According to patient monitoring device 200 shown in FIG. 4C, the test arrangement includes preferably inserting electromagnetic radiation sensor 120 in test port 290, e.g., prior to electromagnetic radiation sensor 120 being coupled in the first arrangement of dressing 110. Preferably, collected electromagnetic radiation 126 in the test arrangement includes a portion of emitted electromagnetic radiation 122 that is redirected by an optically standard material disposed in test port 290. According to one embodiment, the optically standard material preferably includes Spectralon®, manufactured by Labsphere, Inc. (North Sutton, N.H., US), or another material having high diffuse reflectance. Preferably, collected electromagnetic radiation 126 is collected by detector face 142 and the corresponding light signal is transmitted via detection waveguide 140 and plug 170 to patient monitoring device 200. The light signal is preferably compared with accepted calibration values. A satisfactory comparison preferably results in an affirmative indication by at least one of indicator set 240 and display 250; whereas, display 250 may present instructions for additional diagnostic routines and/or guidance for recalibrating or repairing system 200 if the result is an unsatisfactory comparison.

The test arrangement shown in FIG. 4C is preferably a generally passive system for verifying the operation and calibration of system 100. According to other embodiments of patient monitoring device 200, an active testing system preferably includes a light detector to measure the power of emitted electromagnetic radiation 122 and a light source to mimic collected electromagnetic radiation 126.

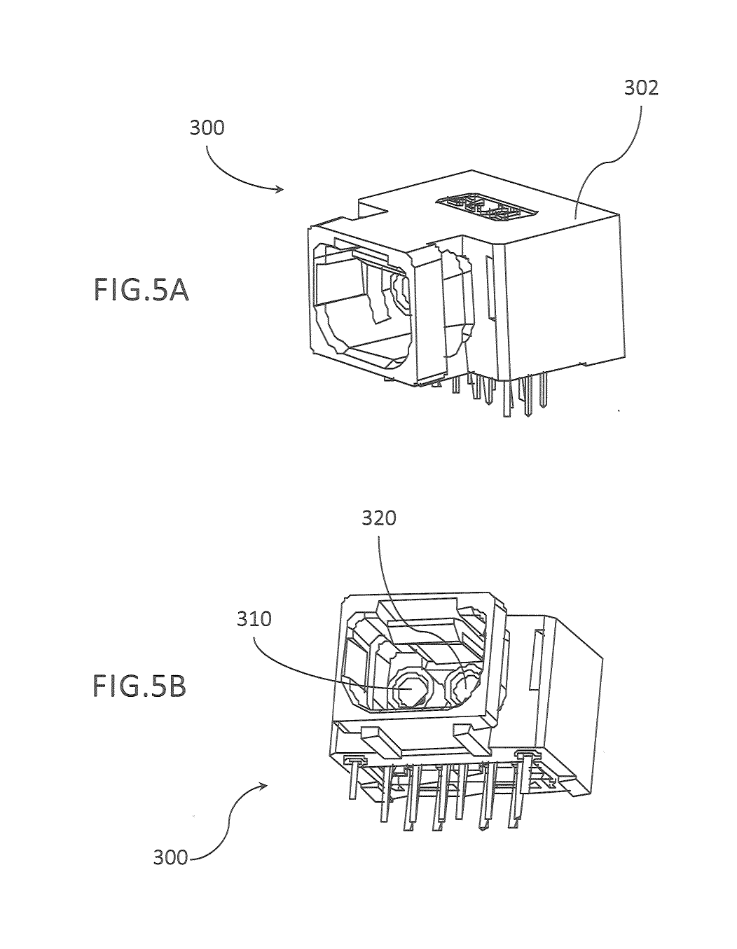

Transfer of the first and second light signals between sensor cable 150 and patient monitoring device 200 is reliably and consistently achieved preferably because of the cooperative engagement between plug 170 and a transceiver 300. Referring additionally to FIGS. 5A and 5B, transceiver 300 is preferably supported by housing 210. According to one embodiment, transceiver 300 is disposed on a bottom surface of housing 210 in generally vertical alignment with test port 290. Preferably, plug 170 and transceiver 300 include mating features that allow repeated coupling and recoupling without substantially affecting the transference of the first and second light signals between sensor cable 150 and patient monitoring device 200. The Small Multimedia Interface (SMI) POF cable assembly plug and SMI TH socket, which are manufactured by Electronic Links International Inc. (Binghamton, N.Y., US), preferably illustrate examples of suitable mating features for plug 170 and transceiver 300, respectively.

Transceiver 300 preferably includes a light-emitting diode 310 and a photodiode 320 supported by an enclosure 302. Preferably, light-emitting diode 310 converts a first electric signal to the first light signal, which yields emitted electromagnetic radiation 122, and photodiode 320 converts the second light signal, which is derived from collected electromagnetic radiation 126, to a second electric signal. According to one embodiment, enclosure 302 segregates light emitting diode 310 and photodiode 320 to preferably eliminate or substantially minimize crosstalk between the first and second light signals as well as between the first and second electric signals. Preferably, enclosure 302 includes an electromagnetic radiation absorbing material to eliminate or substantially minimize electromagnetic interference from outside enclosure 302. According to other embodiments, a filler is preferably injected into enclosure 302 to substantially occupy interior voids and/or occlude exterior gaps. Preferably, the filler includes an electromagnetic radiation absorbing material to eliminate or substantially minimize electromagnetic interference from outside enclosure 302.

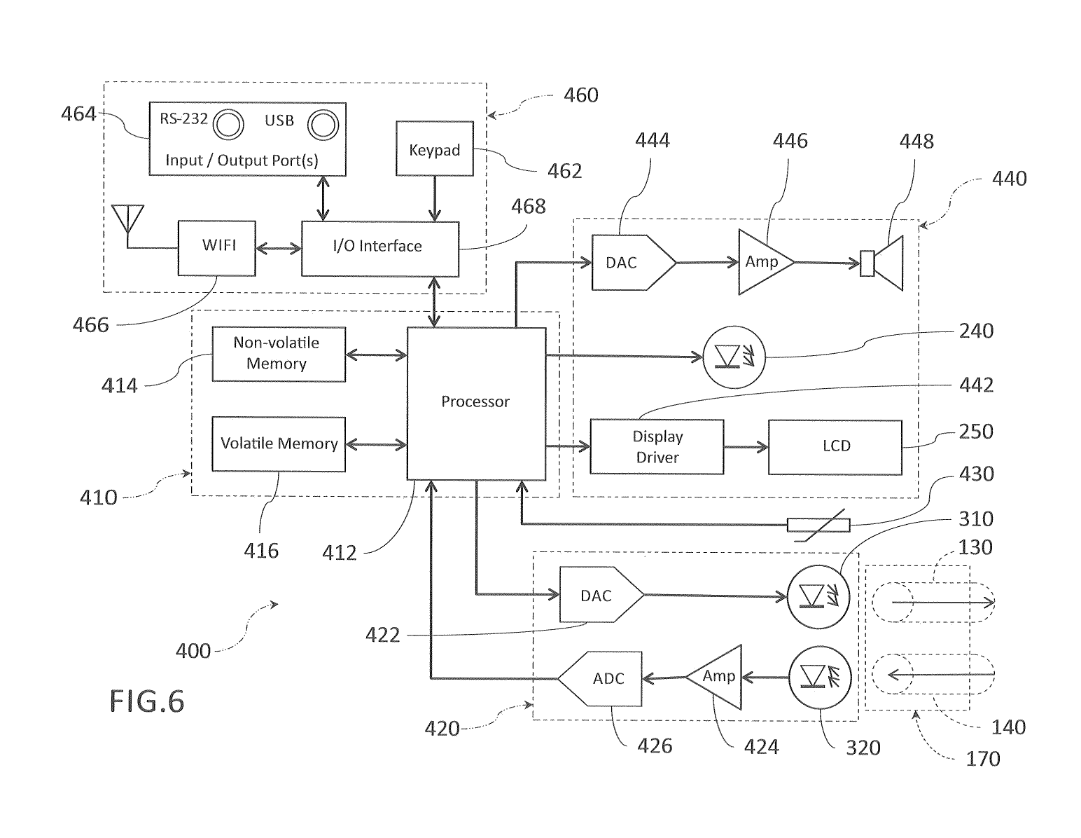

FIG. 6 shows a schematic block diagram of an operating device 400 according to one embodiment of patient monitoring device 200. Preferably, operating device 400 includes a controller 410, at least one optics bench 420, a notification section 440, and an input/output section 460. Controller 410 preferably is disposed in the interior space of housing 210 and includes a processor 412, non-volatile memory 414, and volatile memory 416. According to one embodiment, processor 412 preferably includes a Peripheral Interface Controller (PIC) microcontroller. An example of a suitable processor 412 is model number P132MX695F512L-80I/PT manufactured by Microchip Technology Inc. (Chandler, Ariz., US). Non-volatile memory 414 preferably includes flash memory or a memory card that is coupled with processor 412 via a bi-directional communication link, e.g., a system bus. Preferably, non-volatile memory 414 augments non-volatile memory available on processor 412. According to one embodiment, non-volatile memory 414 includes a Secure Digital (SD) memory card. Volatile memory 416 preferably includes, e.g., random-access memory (RAM), that is coupled with processor 412 via a bi-directional communication link, e.g., the system bus. Preferably, volatile memory 416 augments volatile memory available on processor 412. Preferably, controller 410 performs a number of functions including, inter alio, (1) storing raw data that is collected via sensor 120; (2) processing the raw data according to an algorithm running on processor 412; and (3) storing processed data. According to one embodiment, a timestamp is preferably stored with individual units of raw data, processed data and/or log events. Preferably, controller 410 maintains a log of events related to patient monitoring device 200.

Optics bench 420 preferably is disposed in the interior space of housing 210 and includes a pair of electro-optical signal transducers. Preferably, a first electro-optical signal transducer of optics bench 420 includes a digital-to-analog converter 422 and light-emitting diode 310 to transform a digital electric signal, e.g., the first electric signal, from controller 410 to emitted electromagnetic radiation 122. A second electro-optical signal transducer of optics bench 420 preferably includes photodiode 320, an operational amplifier 424 and an analog-to-digital converter 426 to transform collected electromagnetic radiation 126 to a digital electric signal, e.g., the second electric signal. Preferably, the second electric signal includes the raw data that is collected by controller 410. According to one embodiment, transceiver 300 is preferably supported on a printed circuit board (not shown) that also supports digital-to-analog converter 422, operational amplifier 424 and analog-to-digital converter 426. Preferably, the above mentioned system bus provides communication between optics bench 420 and controller 410. Operating device 400 shown in FIG. 6 shows a single optics bench 420 coupled with controller 410; however, a plurality of optics benches 420 may be coupled with controller 410 when, for example, it is preferable to use a single patient monitoring device 200 with a plurality of electromagnetic radiation sensors 120.

Optics bench 420 preferably operates at low power levels. According to one embodiment, the optical power output of emitted electromagnetic radiation 122 is less than approximately 5 milliwatts and preferably approximately 2 milliwatts. The electrical power output of photodiode 320 derived from collected electromagnetic radiation 126 is generally less than 100 nanoamperes and preferably approximately 2 nanoamperes to approximately 50 nanoamperes.

Patient monitoring device 200 preferably includes a temperature sensor 430 to measure temperature changes that affect light-emitting diode 310. Typically, the intensity of light emanating from light-emitting diode 310 is affected by ambient temperature changes. This accordingly affects the intensity of emitted electromagnetic radiation 122. Temperature sensor 430 preferably measures the ambient temperature and provides to controller 410 an electrical signal that may be used to adjust the electrical signal supplied to digital-to-analog converter 422. Accordingly, the optical power output of light-emitting diode 310 may be generally maintained at a preferable level regardless of ambient temperature changes. According to one embodiment, temperature sensor 430 is preferably disposed in enclosure 302 in proximity to light-emitting diode 310. According to other embodiments, temperature sensor 430 is preferably supported on the printed circuit board for optics bench 420. According to other embodiments, temperature sensor 430 is preferably disposed on a printed circuit board for controller 410. According to other embodiments, temperature sensor 430 is supported on the exterior of housing 210.

Notification section 440 provides visual or audible indications preferably to describe the status of system 100 or to alert a healthcare giver to perform an infiltration/extravasation evaluation. Preferably, visual indicators in notification section 440 include indicator set 240 and display 250. Display 250 is preferably coupled to controller 410 via a display driver 442. An audible indicator preferably includes a digital-to-analog converter 444, an audio amplifier 446, and a speaker 448. Preferably, the digital-to-analog converter 444 communicates with controller 410 via the system bus. Audio amplifier 446 preferably drives speaker 448. According to one embodiment, the output from speaker 448 includes at least one of a tone, a melody, or a synthesized voice. The embodiment of operating device 400 shown in FIG. 6 includes a pair of visual indicators and a single audible indicator; however, other combinations of visual and audible indicators are envisioned.

According to one embodiment, a graphical user interface preferably includes certain features of notification and input/output sections 440 and 460. Preferably, the graphical user interface combines in a generally common area on the exterior of housing 210 at least one of indicator set 240 and display 250 with at least one of soft keys 260, mute button 270, and check button 280. For example, the patient monitoring device 200 shown in FIG. 4A includes a graphical user interface that combines, inter alio, labels 252 on display 250 with soft keys 260.

Input/output section 460 preferably facilitates inputting commands to operating device 400 or outputting data from operating device 400. Preferably, input/output section 460 includes a keypad 462, at least one input/output port 464 or wireless communication device 466, and an input/output interface 468 to couple keypad 462, port(s) 464, and device 466 to controller 410 via the system bus. According to one embodiment, keypad 462 includes soft keys 260, mute button 270, and check button 280. According to other embodiments, keypad 462 preferably includes a keyboard or a touchscreen. According to other embodiments, commands to operating device 400 are preferably input via a pen device or voice recognition device. Input/output ports(s) 464 preferably include connections for communicating with peripheral devices according to at least one standard. Examples of suitable communication standards preferably include, e.g., RS-232 and Universal Serial Bus (USB). Wireless communication device 466 preferably provides an additional or alternate means for communicating with a peripheral device. The embodiment of input/output section 460 shown in FIG. 6 includes three communication options; however, more or less than three options are also envisioned for operating device 400 to communicate with peripheral devices.

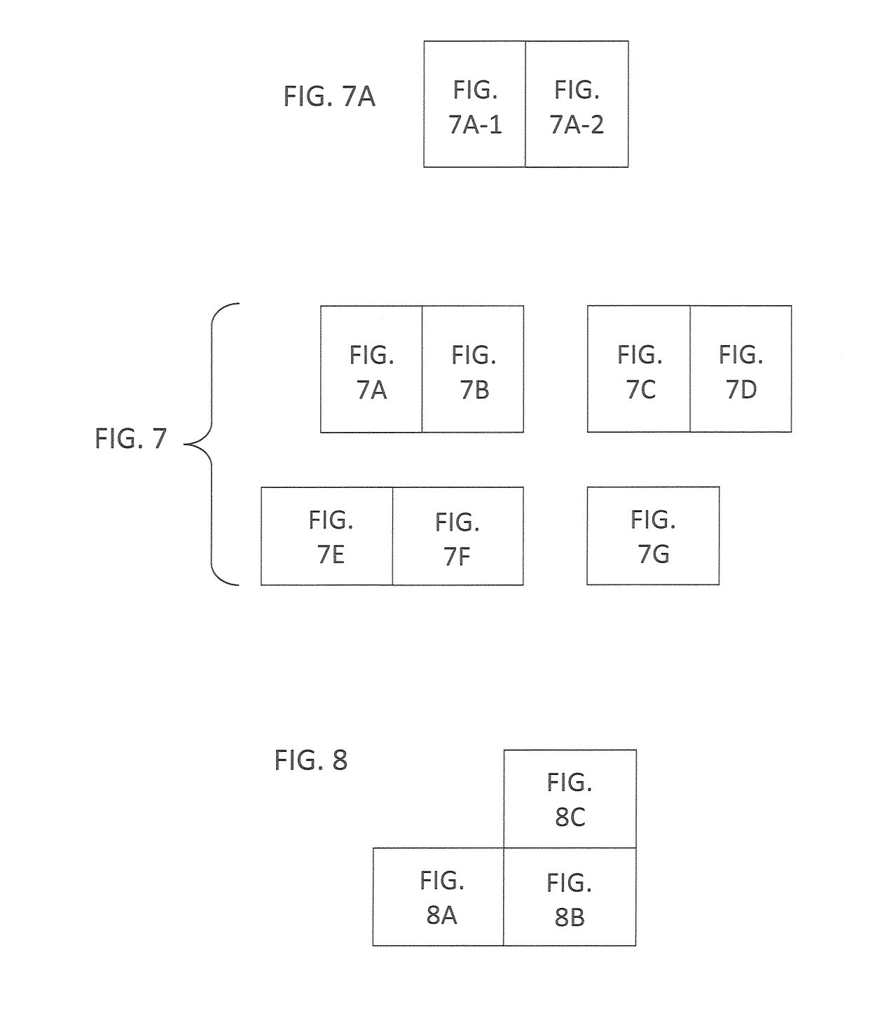

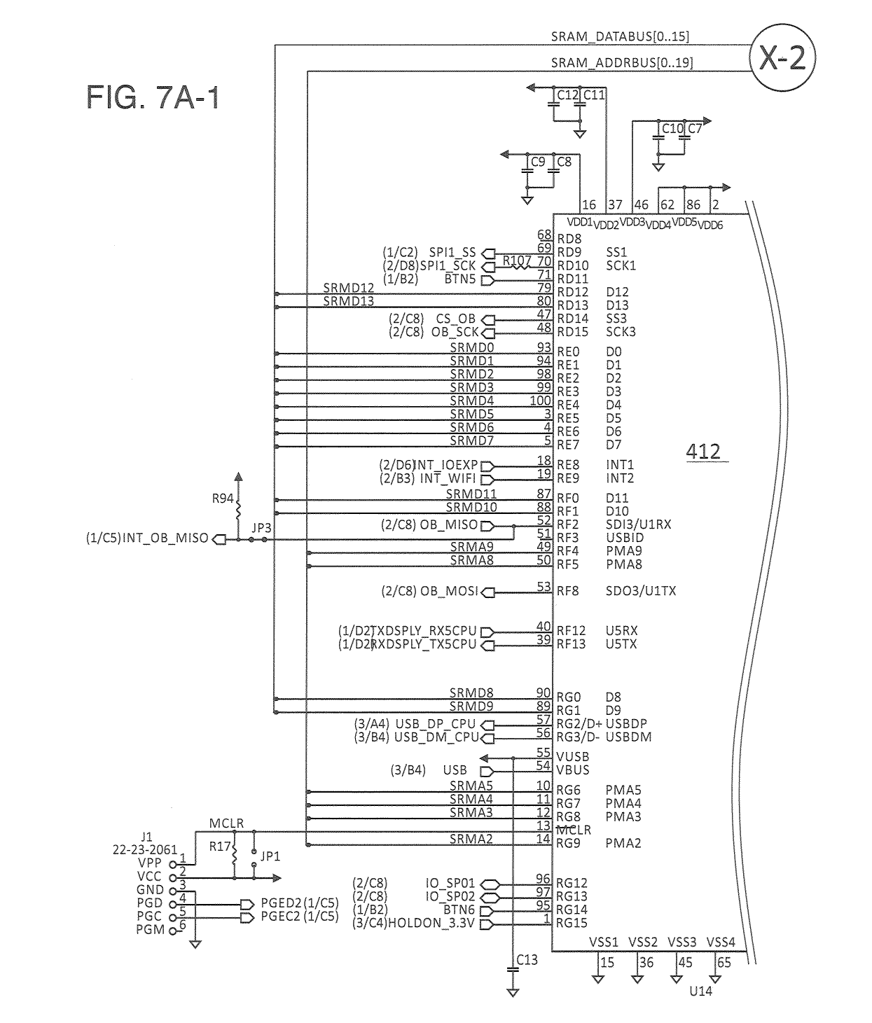

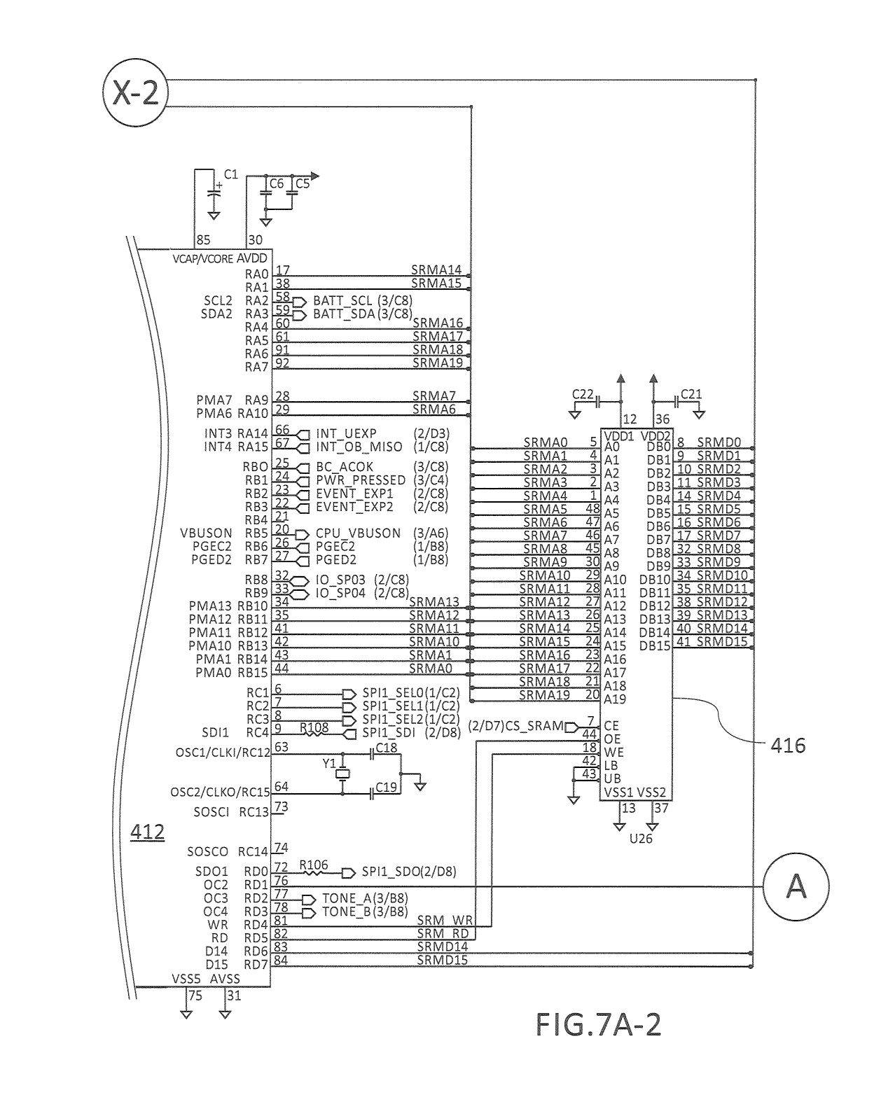

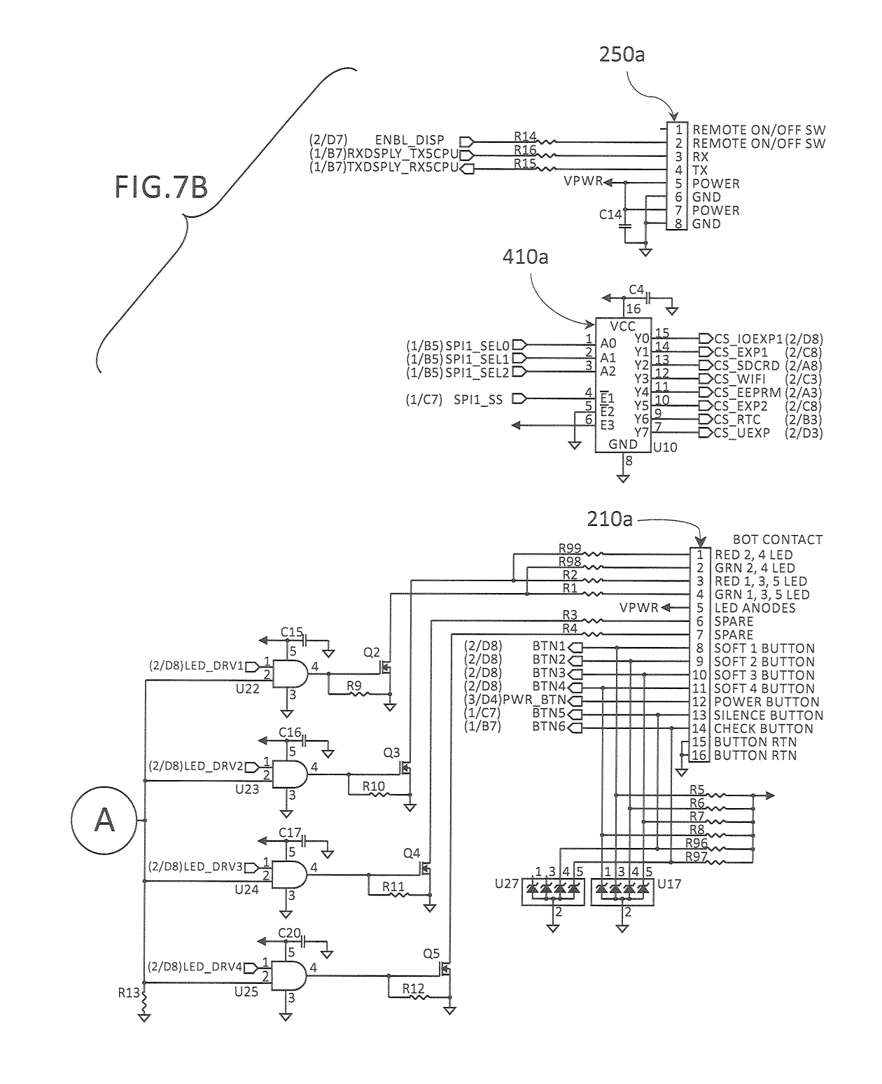

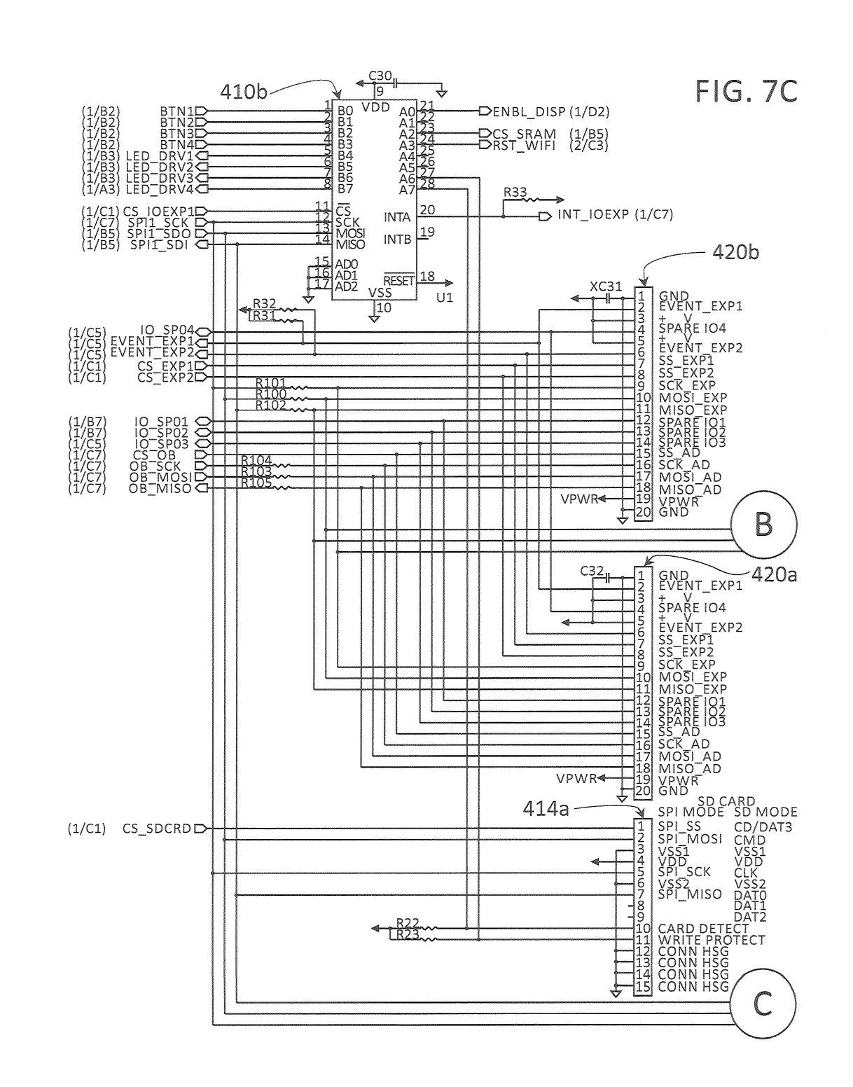

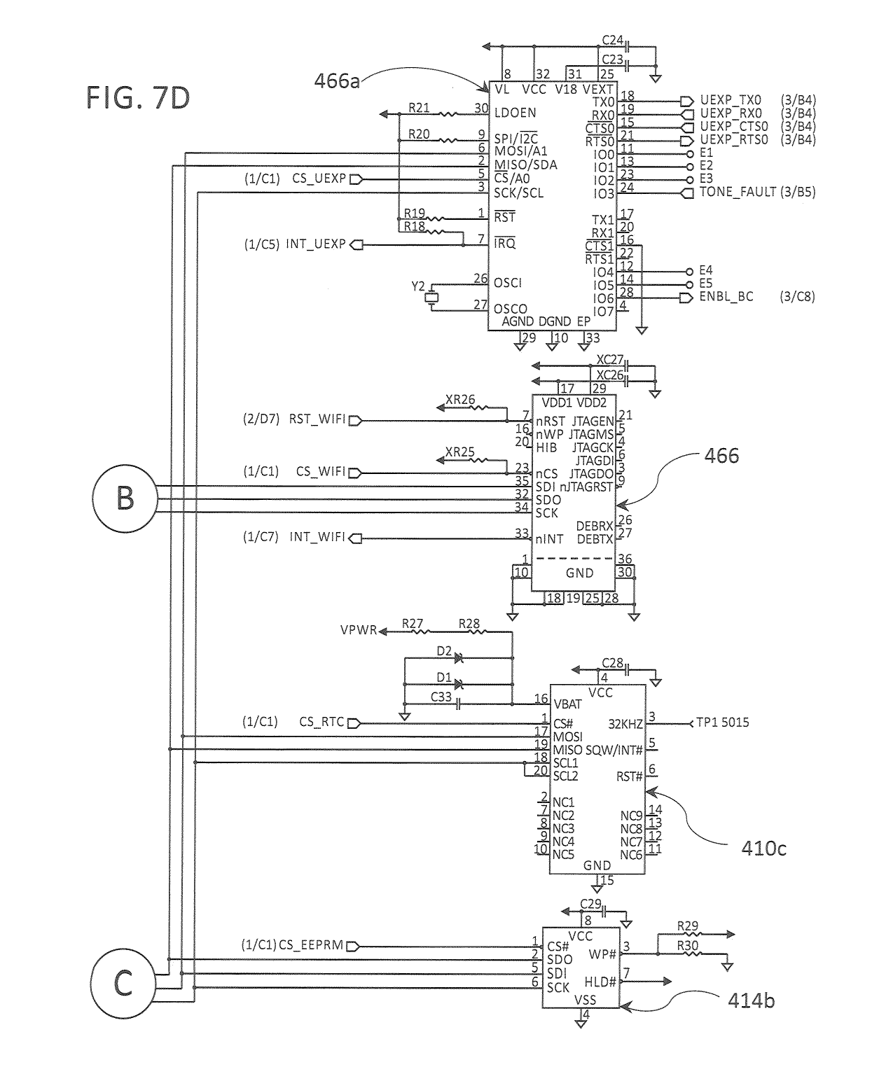

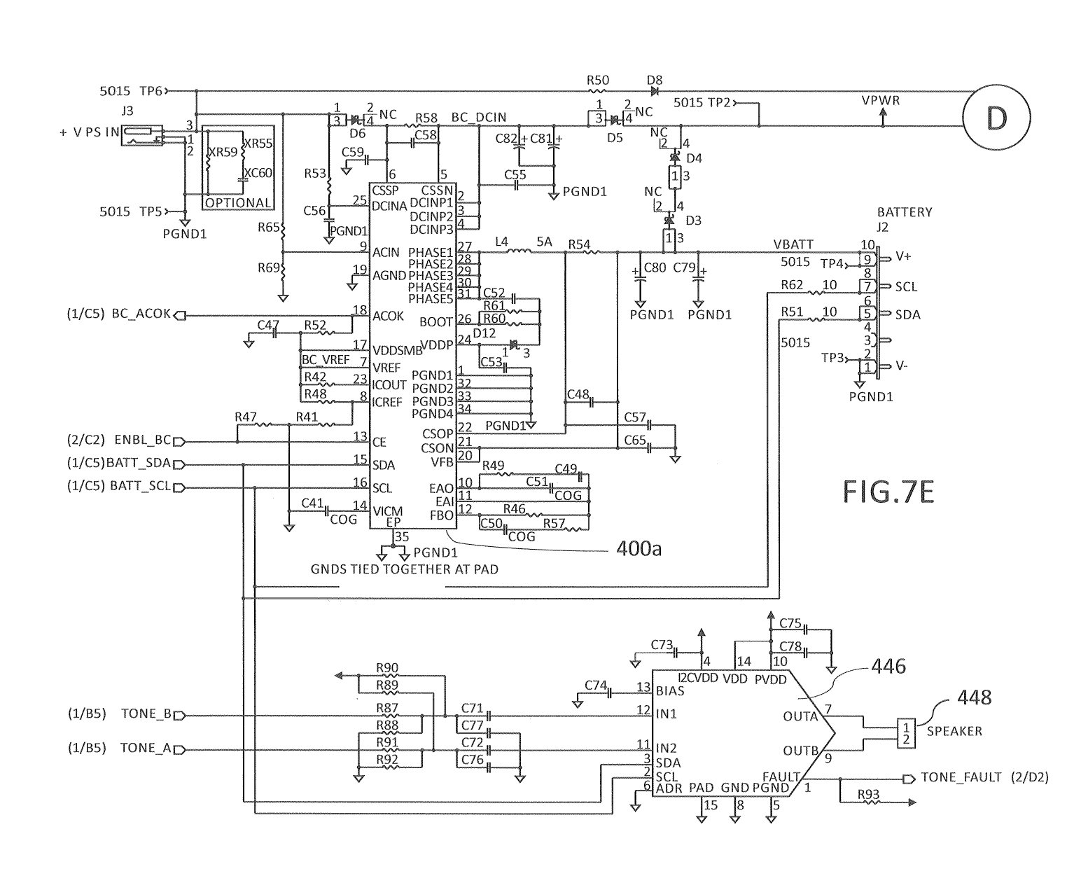

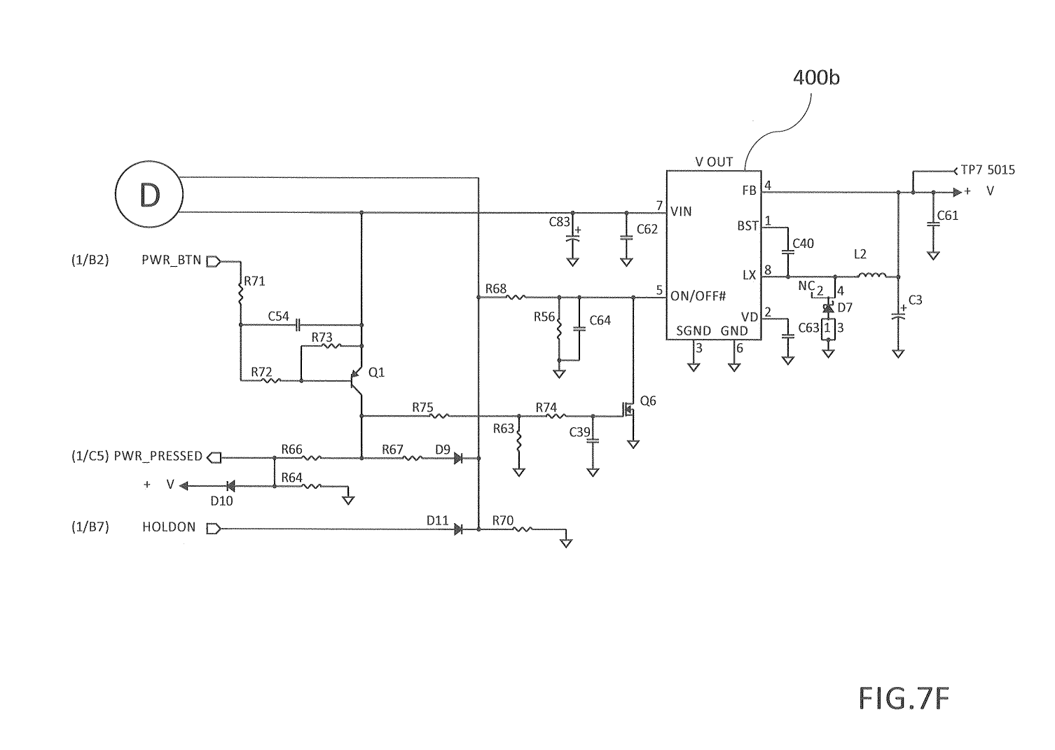

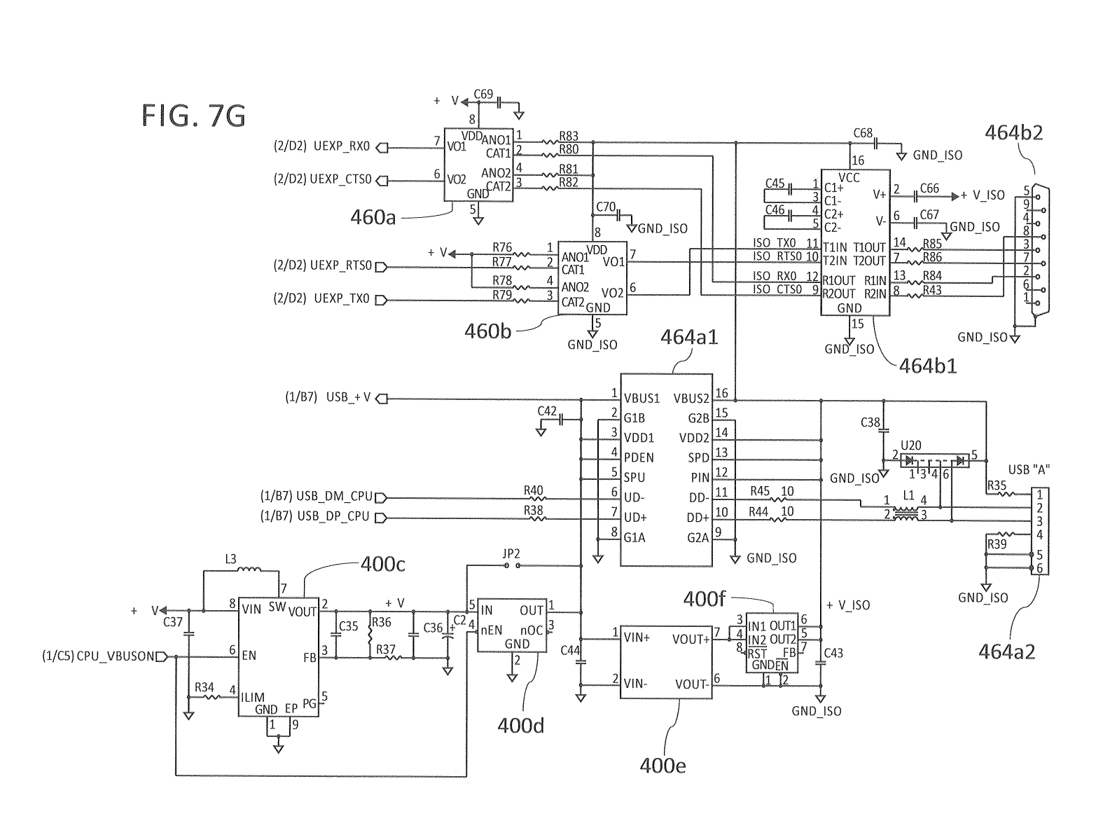

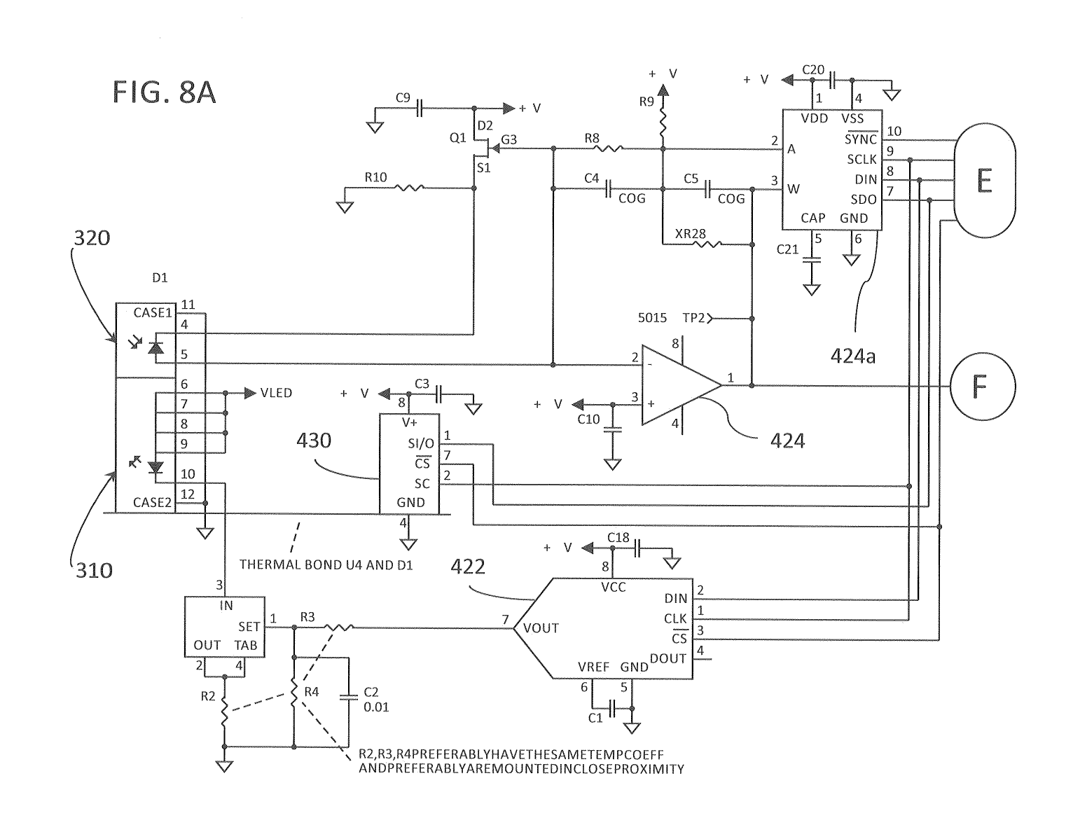

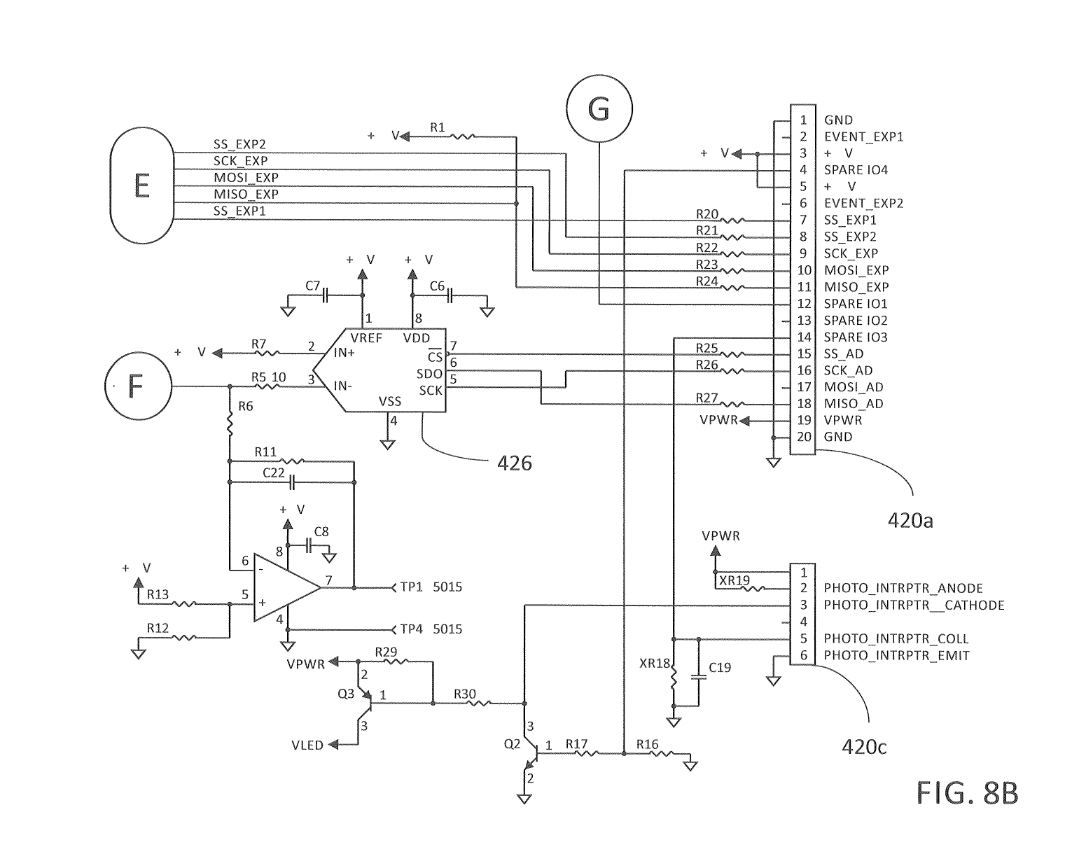

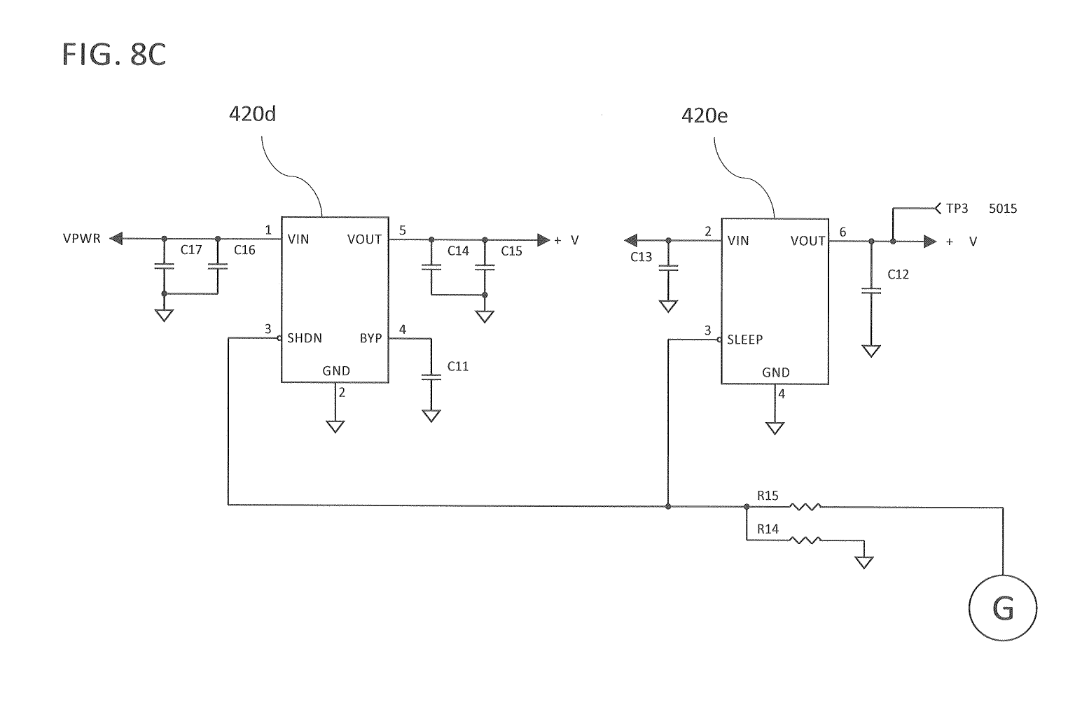

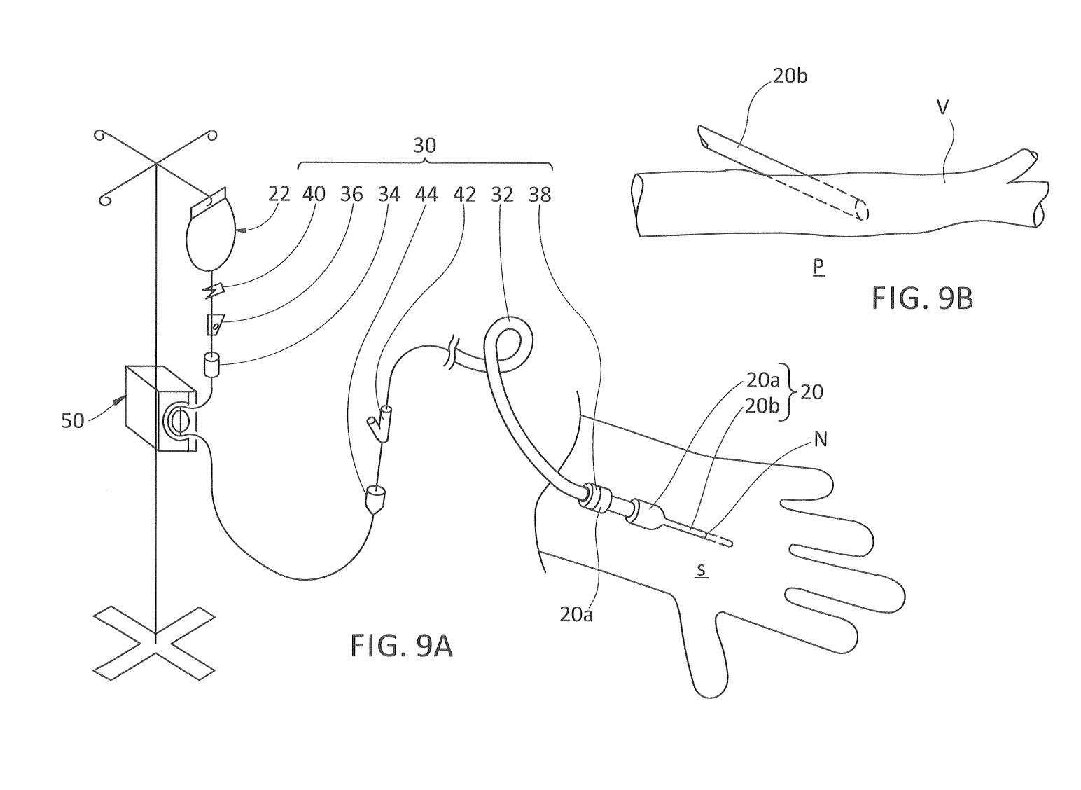

FIGS. 7A-7G and 8A-8C schematically illustrate a circuit diagram of operating device 400 according to one embodiment of the patient monitoring device shown in FIGS. 4A-6. The circuit diagram has been broken into parts and shown on multiple sheets to facilitate understanding; matching letters indicate the connections for the breaks across the sheets. Other connections across sheets are identified with matching labels. FIG. 7A shows preferable electrical connections with respect to processor 412 and volatile memory 416. Preferably, FIG. 7B shows (1) electrical connector 250a for coupling display 250 in operating device 400; (2) electrical connections for a first input/output expander 410a; and (3) electrical connector 210a for coupling operating device 400 with the buttons and indicators supported on housing 210. FIG. 7C shows preferable electrical connections for a second input/output expander 410b, and electrical connections 414a, 420a and 420b for coupling operating device 400 with, respectively, non-volatile memory 414, primary optics bench 420 and an additional optics bench (not shown). Preferably, electrical connection 414a includes a slot for the SD card. FIG. 7D shows preferable electrical connections for an expansion communication port 464a, wireless communication device 466, a clock 410c and an erasable programmable read only memory (EPROM) 414b. According to one embodiment, wireless communication device 466 preferably includes a radio frequency (RF) communicator and expansion communication port 464a may include another wireless communicator, e.g., an infrared communicator. Clock 410c is preferably a remote substitute for a clock available on processor 412. Preferably, EPROM 414b augments the non-volatile memory available on processor 412. FIG. 7E shows preferable electrical connections for a power control 400a, audio amplifier 446 and speaker 448. Preferably, power controls 400a-400f supply and regulate electrical power for operating device 400. FIGS. 7F and 7G show preferable electrical connections for additional power controls 400b-400f. FIG. 7G also shows preferable electrical connections for opto-couplers 406a and 460b, a USB communicator 464a1, and an RS-232 communicator 464b1. Preferably, opto-couplers 406a and 460b shield communication port(s) 464 from damage, e.g., excess voltage or reverse current. According to one embodiment, a USB “A” connector is coupled with USB communicator 464a1 and RS-232 communicator 464b1 is coupled with a DB-9 connector 464b2.

Optics bench 420 according to the embodiment shown in FIGS. 8A-8C illustrates preferred electrical connections for light-emitting diode 310, photodiode 320, digital-to-analog converter 422, operational amplifier 424, and analog-to-digital converter 426. Preferably, operational amplifier 424 is adjusted by a gain control 424a and electrical connector 420a couples optics bench 420 with operating device 400. According to one embodiment, electrical connector 420c couples optics bench 420 with an active test port 290. Power controllers 420d and 420e preferably supply and regulate electrical power for optics bench 420.

Operating device 400 preferably performs a number of functions including emitting the first light signal, detecting the second light signal, transforming the second light signal to a digital signal, and processing the digital signal according to an algorithm. Preferably, the first light signal is emitted by light-emitting diode 310 and the second light signal is detected by photodiode 320. According to one embodiment, controller 410 flashes light-emitting diode 310 periodically. Preferably, the output of photodiode 320 preferably is amplified by operational amplifier 424 and transformed to a digital signal, e.g., a sequence of numerical values, by analog-to-digital converter 426. Processor 412 preferably communicates with volatile memory 416 while evaluating the digital signal, e.g., to identify anatomical changes over time of the structure of the Animalia body or to aid in diagnosing at least one of infiltration and extravasation in Animalia tissue. Preferably, notification section 440 provides at least one of a visual or audible alert when the evaluation performed by processor 412 identifies (1) an anatomical change; or (2) the need for a healthcare giver to perform an infiltration/extravasation evaluation. According to one embodiment, patient monitoring device 200 performs a diagnostic test to verify the calibration of system 100 including electromagnetic radiation sensor 120, sensor cable 150, plug 170, transceiver 300, and optics bench 420. Preferably, non-volatile memory 414 stores the sequence of numerical values along with a corresponding timestamp. The data stored in non-volatile memory 414 preferably is transferred by input/output section 460 from patient monitoring device 200 to a peripheral device, e.g., a patient electronic health record. According to one embodiment, input/output section 460 preferably supplies a control signal to a peripheral device, e.g., infusion pump 50.

While the present invention has been disclosed with reference to certain embodiments, numerous modifications, alterations, and changes to the described embodiments are possible without departing from the sphere and scope of the present invention, as defined in the appended claims. For example, certain sections of the operating system may use different voltages or the operating system may use a common voltage. For another example, the source of electric power for operating system may be internal, e.g., a battery, or external, e.g., alternating current and an alternating to direct current converter. Accordingly, it is intended that the present invention not be limited to the described embodiments, but that it has the full scope defined by the language of the following claims, and equivalents thereof.

What is claimed is:

1. A device to aid in diagnosing at least one of infiltration and extravasation in Animalia tissue, the device comprising:
an optics bench including a light emitting diode and a photodiode, the light emitting diode being configured to emit a first light signal, and the photodiode being configured to detect a second light signal, the second light signal including a portion of the first light signal that is at least one of reflected, scattered and redirected from the Animalia tissue;
a controller including volatile memory and a processor, the volatile memory being configured to store a sequence of values corresponding to the second light signal detected by the photodiode, and a processor being configured to analyze the sequence of values stored in the volatile memory; and
an indicator coupled to the processor and being configured to output a notice regarding at least one of infiltration and extravasation in the Animalia tissue.

2. The device of claim 1 wherein the optics bench includes a transceiver fixing the light emitting diode with respect to the photodiode.

3. The device of claim 2 wherein the transceiver comprises a fitting configured to cooperate with a mating feature of a plug, and the light emitting diode and photodiode are configured to align with end faces of optical fibers fixed in the plug.

4. The device of claim 1 wherein the first light signal emitted by the light emitting diode consists of near-infrared light, and the second light signal detected by the photodiode consists of near-infrared light.

5. The device of claim 1 wherein wavelengths of the first and second light signals are between approximately 600 nanometers and approximately 1,800 nanometers.

6. The device of claim 1 wherein the first and second light signals are centered about approximately 940 nanometers.

7. The device of claim 1 wherein the optics bench comprises a digital-to-analog converter coupled to the light emitting diode and an analog-to-digital converter coupled to the photodiode, the digital-to-analog converter is configured to adjust a first electric signal corresponding to intensity of the first light signal, and the analog-to-digital converter is configured to sample a second electric signal from the photodiode corresponding to intensity of the second light signal.

8. The device of claim 7 wherein the optics bench comprises an amplifier coupling the photodiode and the analog-to-digital converter.

9. The device of claim 8 wherein the amplifier has a gain of approximately 0.5 million volts per ampere to approximately 35 million volts per ampere.

10. The device of claim 1 wherein optical power of the first light signal emitted by the light emitting diode is approximately 2 milliwatts, and electric current corresponding to the second light signal detected by the photodiode is less than approximately 100 nanoamperes.

11. The device of claim 1 wherein optical power of the first light signal emitted by the light emitting diode is approximately 2 milliwatts, and electric current corresponding to the second light signal detected by the photodiode is approximately 2 nanoamperes to approximately 50 nanoamperes.

12. The device of claim 1 wherein the optical bench is disposed on a first printed circuit board, the controller is disposed on a second printed circuit board, and an electric cable couples the first and second printed circuit boards.

13. The device of claim 1 wherein the controller comprises an analog-to-digital converter coupled to the photodiode, the analog-to-digital converter is configured to sample an electric signal from the photodiode corresponding to intensity of the second light signal, and output from the analog-to-digital converter comprises the sequence of values.

14. The device of claim 13 wherein the controller comprises an amplifier configured to amplify the electric signal.

15. The device of claim 14 wherein the amplifier has a gain of approximately 0.5 million volts per ampere to approximately 35 million volts per ampere.

16. The device of claim 1 wherein the processor runs an algorithm operating on the sequence of values.

17. The device of claim 16 wherein the controller comprises non-volatile memory configured to store results of the algorithm.

18. The device of claim 16 wherein the controller comprises a communicator configured to transfer off the device results of the algorithm.

19. The device of claim 1, comprising a graphical user interface coupled to the controller.

20. The device of claim 19 wherein the graphical user interface comprises at least one of (i) a display screen, and (ii) a set of indicator lights.

21. The device of claim 19 wherein the graphical user interface comprises a set of pushbuttons.

22. The device of claim 1, comprising—
a housing generally enclosing the optics bench and the controller;
a power source disposed in the housing, the power source being coupled to at least one of the optics bench and the controller.
